(19) United States
(12) Patent Application Publication
(10) Pub. No.: US 2014/0273010 A1
(43) Pub. Date: Sep. 18, 2014

(54) ACID-CLEAVABLE AND CLICKABLE AFFINITY CAPTURE PROBE

(71) Applicant: The Research Foundation for The State University of New York, Albany, NY (US)

(72) Inventors: Nicole Sampson, Setauket, NY (US); Siyeon Lee, Stony Brook, NY (US)

(73) Assignee: The Research Foundation for The State University of New York, Albany, NY (US)

(21) Appl. No.: 14/212,477

(22) Filed: Mar. 14, 2014

Related U.S. Application Data
(60) Provisional application No. 61/801,397, filed on Mar. 15, 2013.

(51) Int. Cl. G01N 33/53

(52) U.S. Cl. CPC G01N 33/53
USPC 435/7.5; 436/501; 548/304.1

(57) ABSTRACT

The present disclosure relates to biological probes useful for detecting the presence of a target molecule. The biological probes are capable of forming complexes with the target molecule that are stable to reduction, oxidation and hydrolysis.

### CROSS REFERENCE TO RELATED APPLICATIONS

This application claims priority from U.S. Provisional Application No. 61/801,397, filed Mar. 15, 2013, the entire contents of which are incorporated herein by reference.

### STATEMENT REGARDING FEDERALLY SPONSORED RESEARCH

This disclosure was made with government support under grant numbers GM097971 and HD038519 awarded by the National Institutes of Health. The government has certain rights in the disclosure.

### INCORPORATION BY REFERENCE OF SEQUENCE LISTING

The Sequence Listing in the ASCII text file, named 29894_sequencelisting.txt of 1 kilobytes, created on Mar. 14, 2014, and submitted to the United States Patent and Trademark Office via EFS-Web, is incorporated here by reference.

### FIELD OF THE DISCLOSURE

This disclosure relates to an acid sensitive cyclic acetal cleavable affinity tag and the use thereof for labeling proteins.

### BACKGROUND OF THE DISCLOSURE

Identification of proteins within a cellular context that bind a specific ligand, e.g., a drug or an effector molecule, is a challenge in the biological field. The probes must bond to the target protein with sufficient affinity and selectivity that the protein can be isolated, and the capture process cannot cause damage to the molecular structure of the protein. Otherwise, identification becomes quite difficult, if not impossible. If a protein is of low abundance, the challenge is further increased.

A typical strategy is to prepare a bifunctional probe. One end of the probe molecule contains the ligand that binds to the target molecule, i.e., the protein. The other end of the ligand molecule contains a moiety which enables capture and isolation of the ligand-protein complex. However, modification of the ligand molecule to contain a moiety which enables capture and isolation of the ligand-protein complex can dramatically alter binding affinity and specificity. Therefore, a better approach is to incorporate a small chemical handle onto the ligand to which the capture reagent can be covalently attached after binding to the receptor. The complex is captured typically on a bead matrix that permits easy separation of the complex from the remainder of the cellular debris. Once captured, the protein of interest must be selectively removed from the bead matrix in order to submit to analytical analysis, usually mass spectrometry, for identification purposes. Alternatively, the aldehyde that is unmasked during acid cleavage may be used to further incorporate molecular functionality onto the targeted protein.

The azide-alkyne cycloaddition “click” reaction has been widely employed for covalent modification in biological studies. The azide and alkyne form a triazole in the presence of Cu(I) catalyst, and the resulting triazole is stable to further reaction conditions such as reduction, oxidation, and hydrolysis. Fast and orthogonal covalent modification of proteins is very important since identification and analysis of targeted proteins can only be achieved with a tag at a specific site. For this reason, the azide-alkyne cycloaddition has been chosen for many biological studies, e.g. selective protein modification by site directed mutagenesis in vitro and in vivo, and activity-based protein profiling.

Biotin is often used to capture the targeted protein due to its strong binding affinity for the egg-white glycoprotein avidin or to the bacterial protein streptavidin. Enrichment of biotinylated proteins from a complex mixture is efficiently achieved using a streptavidin-coated solid support such as an agarose resin. Combined with alkyne-azide cycloaddition, which enables the coupling of probes to targeted proteins, biotin tags linked to an alkyne or azide have become a powerful tool for purification and analysis of proteins in proteomics.

However biotin, as well as other high affinity ligands, often creates a problem. Biotin requires harsh elution conditions to release the captured protein from the bead matrix. Conventional methods to release biotinylated proteins from streptavidin bead matrices are harsh because of the strong binding interaction. For example, 2% SDS/6M urea, boiling in 2% SDS or on-bead tryptic digestion are required to release the targeted protein. In addition to eluting the desired protein, these non-selective conditions release streptavidin monomer from the matrix, or undesired intrinsically biotinylated proteins, or on-bead digestion results in elution of proteolyzed peptides from strepavidin after trypsin treatment. All of these elution methods lead to contamination of the protein to be analyzed. As a consequence of the difficulty in isolating only the desired protein from the matrix, the mass spectra required for target identification have increased noise.

Recently, several biotin probes containing cleavable linkers have been developed to avoid such harsh elution conditions. Disulfide linkers have been widely used due to their rapid cleavage under mild reducing conditions. However, a disulfide linker is unstable to electrophilic and nucleophilic polar reagents, and thiol exchange with thiols in biological fluids can occur. Photocleavable linkers use long-wave UV light to cleave the linker, but in some conditions, illumination of the sample is limited. There is also an acid labile linker from Pierce (proprietary structure) that is cleaved in 95% TFA. But, TFA is a strong carboxylic acid. Removal of this linker with 95% TFA may cause release of non-specifically captured proteins or the streptavidin itself. Another alternative is a dialkoxydiphenylsilane linker that is reported to be efficiently cleaved upon treatment with 10% formic acid for 0.5 h. The silane is sterically large and can hinder reaction with the desired target. Thus, there is a need for a probe that is not only easy to synthesize but also is easy to cleave from the protein under mild acidic conditions. The present disclosure accomplishes that goal.

Use of the cyclic acetal provides an aldehyde tag on the captured protein after acid cleavage. This aldehyde tag may be used for further modification, e.g., for incorporation of a fluorophore, radiolabel or an isotopic mass tag.

Variations of the probe that include different click moieties or different capture moieties, are thus readily accessible.

### SUMMARY OF THE DISCLOSURE

The present disclosure is directed to a biological probe of the formula:

Q-L₁-X₂—X₃—R₂—R₃-L₂-Y I

wherein

[chemical structure]

X₁, X₂ and X₃ are independently absent, a bond, C(R₁)₂, NR₁, O, S, or CO;
each R₁ is the same or different and is H, or (C₁-C₁₀) alkyl;
L₁ is a bond, (C₂-C₅₀ alkyl), polyethylene glycol of the formula CH₂CH₂—(O—CH₂CH₂)n, wherein n is 1-30, a polypeptide of 1-15 amino acids of the formula AA₁-AAₙ₁, or a polypeptide of 1-15 amino acids of the formula: AAₘ-AA₁;
each AA is the same or different amino acid,
m and n₁ are independently 0-14.
R₂ is C₁-C₃₀ alkyl, disubstitued aryl, disubstituted arylalkyl, or
polyethylene glycol of the formula CH₂CH₂—(O—CH₂CH₂)ₙ₂ wherein n₂ is 1-30;

### R₃ is

[chemical structure]

L₂ is a bond, X₄-T-X₅, wherein T is an alkyl group of C₂-C₅₀, or a polyethylene glycol of the formula X₄—CH₂—CH₂—(O—CH₂—CH₂)n-X₅

### X₄ is C(R₅)(R₆), CO;

X₅ is N(R₇), O, or S or a polypeptide of the formula AA₁-AAₙ₃
n₃ is 0-14
R₅, R₆, and R₇ and are independently H or C₁-C₁₀ alkyl,

[chemical structure]

or an affinity tag peptide,
with the proviso that there can be no two adjacent oxygen, sulfur, nitrogen or carbonyl atoms, and when L₁ is a polypeptide of the formula AA₁-AAₙ₁, then X₂ is a bond and AA₁ is bonded to X₁ and AAₙ₁ is bonded to X₃ or when L₁ is a polypeptide of the formula: AAₘ-AA₁, then X₂ is a bond and AA₁ is bonded to X₃ and AAₘ is bonded to X₁.

An embodiment of the present disclosure is directed to a compound of the formula

[chemical structure]

In addition, the present disclosure is directed to a method for detecting the presence of a target molecule by (a) reacting the probe of the present disclosure with a target protein having an alkyne moiety thereon under click reaction conditions to form a probe-complex; (b) immobilizing the probe complex onto a stationary support; (c) removing the probe complex from the support; and (d) analyzing the target protein.

### BRIEF DESCRIPTION OF THE DRAWINGS

These and other features, aspects, and advantages of the present disclosure will become better understood with regard to the following description, appended claims, and accompanying drawings wherein:

FIG. 1 evaluates the cleavable biotin probe of the present disclosure and compares the results with non-cleavable probe. FIG. 1A is a Streptavidin blot where, each biotin probe was incubated in 1% TFA at 37° C. for different duration time without streptavidin-resin. Lane 1: alkyne functionalized BSA; lane 2, modified with probe 7a; lane 3, modified with probe 7b; lane 4: modified with probe 8; lane 5-7, same order as lanes 2-4; lanes 8-10, same order as lanes 2-4.

FIG. 1B shows the Ponceu staining of the cleavage test with streptavidin-resin. Lane 1 and 2: boiling the beads that captured BSA modified by probe 7b and 8, respectively before cleavage; lane 3-4, supernatant after incubating the loaded resin in cleavage condition; lanes 5-6, boiling the resin after cleavage.

FIG. 2A provides the results that show the comparison of BSA-7b and BSA-7c linker cleavage. Lane 1, BSA-7b captured on streptavidin-agarose beads; Lane 2, BSA-7c captured on streptavidin-agarose beads; lane 3, supernatant from BSA-7b incubated in 1% TFA at 37° C. for 1 hour; lane 4, supernatant from BSA-7c incubated in 1% TFA at 37° C. for 1 hour; lane 5, streptavidin-agarose beads with captured BSA-7b after TFA incubation; lane 6, streptavidin-agarose beads with captured BSA-7c after TFA incubation.

FIG. 2B provides the results of capture and cleavage test of BSA-7c in a bacterial whole cell lysate. Lane 1, cell lysate+BSA-7c; lane 2, BSA-7c captured on streptavidin-agarose beads from cell lysate; lane 3, supernatant from BSA-7c incubated in 1% TFA at 37° C. for 1 hour; lane 4, streptavidin-agarose beads with captured BSA-7c after TFA incubation.

FIG. 3 provides the results of RNase A-7c capture and elution. FIG. 3A is the Ponceu staining of the capture of RNase A-7c in the presence of bacterial whole cell lysate and release; lane 1, RNase A-maleimide alkyne; lane 2, RNase A-7c; lane 3, cell lysate+RNase A-7c; lane 4, supernatant after RNase A-7c streptavidin capture; lane 5, RNase A-7c captured on streptavidin-agarose beads from cell lysate; lane 6, supernatant from RNase A-7c incubated in 1% TFA at 37° C. for 1 hour; lane 7, streptavidin-agarose beads with captured RNase A-7c after TFA incubation. FIG. 3B is the Ponceu staining of streptavidin monomer release under cleavage conditions used for other cleavable linkers; lane 1, Na₂S₂O₄ for 1 hour at 25° C.; lane 2, 2% 2-mercaptoethanol for 1 h at 25° C.; lane 3, 5% formic acid for 2 hours at 25° C.; lane 4, 1M guanidine hydrochloride in 1% TFA at 37° C. for 1 hour. FIG. 3C is the Ponceu staining and Streptavidin blot of the effect of guanidine concentration on streptavidin monomer release from the beads; lane 1, cell lysate+RNase A-7c; lane 2, supernatant after RNase A-7c streptavidin capture; lane 3, RNase A-7c captured on streptavidin-agarose beads from cell lysate; lane 4, supernatant from RNase A-7c incubated in 1M guanidine/1% TFA at 37° C. for 1 hour; lane 5, supernatant from RNase A-7c incubated in 3M guanidine/1% TFA at 37° C. for 1 hour; lane 6, streptavidin-agarose beads with captured RNase A-7c after 1M guanidine/TFA incubation; lane 7, streptavidin-agarose beads with captured RNase A-7c after 3M guanidine/TFA incubation.

FIG. 4 provides the Ponceu staining and Streptavidin blot of further modification of the BSA aldehyde tag; lane 1, BSA-7c; lane 2, BSA-7c captured on streptavidin-agarose beads; lane 3, supernatant from BSA-7c incubated in 1% TFA at 37° C. for 1 hour; lane 4, BSA-aldehyde after reaction with alkoxyamine-PEG-biotin at pH 5, 37° C. for 4 hours.

### DETAILED DESCRIPTION OF THE DISCLOSURE

As defined herein, the term alkyl refers to alkyl groups. The number of carbon atoms in the groups is designated in the definitions. Some alkyl groups as defined may have 1-10 carbon atoms, while other alkyl groups may have 1-30 carbon atoms and others may have 2-50 carbon atoms. They may be straight-chained or branched. Examples include methyl, ethyl, i-propyl, n-propyl, n-butyl, isobutyl, sec-butyl, n-pentyl, isopentyl, neopentyl, n-hexyl, n-heptyl, 3-methyhexyl, octyl, nonyl decyl and the like.

AA₁, AAₙ₁ and AAₙ₃ as defined herein are independently amino acids. In one embodiment, the amino acids are α-amino acids. In yet another embodiment, the amino acids are L-α-amino acids. In yet another embodiment, the amino acid is Gly.

As written herein, the term AAₘ-AA₁ and AA₁-AAₙ₃ and AA₁-AAₙ₁ independently refer to a moiety comprised of 1-15 amino acids wherein m, n₁ and n₃ independently are integers of 0-14.

The term “aryl”, as defined herein refers to aromatic atoms wherein the ring atoms in the aromatic group are all carbon atoms. Aryl groups can preferably contain 6, 10 or 14 ring carbon atoms. Examples include phenyl, naphthyl, anthracenyl, and the like. Preferred aryl groups are phenyl.

The compounds of the present disclosure are as defined hereinabove.

As defined, Y can be an affinity tag. Examples include, for example, FLAG tag: DYKDDDDK (SEQ. ID NO.: 1); Strep Tag: WSHPQFEK (SEQ. ID NO. 2); c-Myc Tag: EQKLISEEDL (SEQ ID NO.: 3).

An embodiment of the present disclosure has the formula:

[chemical structure]

The presence of the azide moiety on one end allows the probe to react with the target protein having an alkyne moiety thereon under click reaction conditions. The azide and alkyne form are reacted in the presence of a copper (I) catalyst under Huisgen 1,3-dipolar cycloaddition conditions to form a triazole. The resulting triazole is quite stable to further reaction conditions such as reduction, oxidation and hydrolysis.

The other end of the probe has the biotin functionality or the tag peptide. This permits the probe to be immobilized on a solid support, such as a streptavidin-coated solid support e.g., an agarose resin when Y is a biotin moiety. Thus, for example, while the probe is immobilized on the solid support, the target molecule at the other end can be further analyzed or purified or subjected to further treatment or further manipulations.

But, the advantage of the probe of the present disclosure is that it is easily removed from the support onto which it is immobilized without damaging, modifying or altering the molecular composition of the protein attached to the probe. More specifically, the protein is removed from the support under mild acid conditions, such as very dilute trifluoroacetic acid. In an embodiment, the probe may be released from the support using 1% (v/v) trifluoroacetic acid. Any dilute aqueous acid solution with a pH of approximately 1-2 would be suitable for acetal cleavage.

The conditions to cleave the probe from the support are sufficiently mild so as to minimize release of non-specifically bound protein attached to the streptavidin-coated solid support. Once removed from the support, the desired protein can be further characterized, such as by taking the mass spectrum of said protein. Alternatively, the target protein can be further characterized to determine its molecular or physical properties or tagged with an imaging label. In certain embodiments such labels include, but are not limited to, a fluorescent molecule or molecules, an isotope mass tag, or a radioactive moiety.

The present disclosure contemplates three different acetal moieties. They differ by the type of acetal that is present on the probe. In one embodiment, the acetal present is

[chemical structure]

In another embodiment, the acetal is

[chemical structure]

In another embodiment, the acetal is

[chemical structure]

The probe of the present disclosure is prepared by utilizing chemical techniques known to one of ordinary skill in the art. For example, the compounds of the present disclosure may be prepared by amidation under amide forming conditions, i.e., the reaction of an amine with a carboxylic acid or carboxylic acid derivative, such as an acid halide, wherein the halide is a chlorine or bromine. For instance, the compounds of the present disclosure may be prepared by reacting Q-L₁-X₂—X₃—R₂—R₃—H with YH wherein Q, L₁, X₂, X₃, R₂, R₃ and Y are as defined herein above and L₂ is a bond under amide forming conditions. Alternatively, in another embodiment, Q-L₁-X₂—X₃—R₃—H may react with AAₙ-AA₁ under amide forming conditions, wherein L₁, X₂, X₃ and R₃ are as defined herein and L₂-Y is AA₁-AAₙ-Y optionally in the presence of a coupling agent, such as dicylohexylcarbodiimide. In this embodiment, L2-Y is also prepared under amide forming conditions.

In another embodiment, the compounds of the present disclosure may be prepared by esterification under ester forming conditions. For example, when L₁ is a bond and X₂ is absent, and X₃ is carbonyl and X₁ is O, and Q, R2, R3, L2 and Y are as defined herein above, then the compound of formula I may be prepared by reacting Q-X1-OH with Z—X3-R2-R3-L2-Y wherein Z is OH or a halide wherein halide is bromide or chloride under esterification conditions.

Alternatively, the probe of Formula I may be prepared by nucleophilic substitution reactions. For example, Q-L1-X2-X3H, wherein Q, L1, X2 are as defined herein above and X3 is NH, may be reacted with LG-R2-R3-L2-Y, wherein R3, L2 and Y are as defined herein above and R2 is CH2-Ar, wherein Ar is aryl, LG is a leaving group, such as halide, mesylate, brosylate, tosylate, under nucleophilic substitution reactions in the presence of a strong base, such as sodium hydroxide, potassium hydroxide, sodium hydride and the like.

These synthetic techniques are only exemplary. Variations of the synthetic routes described herein may be utilized to prepare the compounds of the present disclosure. Further different fragments may be bonded together and the compounds of Formula I may be prepared by joining fragment and repeating one or more of these reactions or other types of reactions using the techniques known to one of ordinary skills in the art.

If there are groups on the reactants that are reactive under the conditions of the reaction, then protecting groups can be used to protect those groups.

In an embodiment, the probe of formula I is prepared by the in accordance with the procedure depicted in scheme 1.

[chemical structure]

Azide-PEG₈-amine is coupled to dimethoxy acetal 1 under amide forming conditions to form the amide. The reaction is effected in an appropriate solvent in the presence of a coupling agent, such as carbonyl diimidazole. The product 2 is purified using techniques known to the skilled artisan. For example, since 2 is sensitive to acid, it can be purified using column chromatography with alumina as the adsorbent. Depending upon the type of acetal that is to be formed in the probe, either serinol or 3-amino-1,2-propanediol is the reagent to be reacted with 2. However, since the amine is reactive with the aldehyde, an amine protected serinol or an amine protected 3-amino-1,2 propanediol is reacted with 2 under effective conditions. For example, in an embodiment, depending upon which probe is prepared, the serinol or the 3-amino-1,2-propanediol is protected as their trifluoracetamides, 3 or 4. Next, the cyclic acetal 5 is formed under acetal forming conditions. In an embodiment, it is formed using p-toluene sulfonic acid as a catalyst in THF/toluene. THF is used to dissolve diol 3 or 4. After purifying 5, the trifluoroacetamide is removed to generate free amine for the coupling reaction. Finally, the amine is coupled to biotin under effective conditions to produce the final product, 7a, 7b or 7c.

The simplicity of the chemistry enables the preparation of probes of varying structure in order to identify the probe best suited to the specific target capture application.

The probe is coupled to a protein that has an alkyne moiety thereon using techniques known in the art. More specifically, the coupling is conducted using a Cu(I) catalyst under Hsuigen 1,3-dipolar cycloaddition conditions to effect azide-alkyne cycloaddition “click” reaction so that the azide and alkyne form a triazole. The resulting triazole is stable to further reaction conditions such as reduction, oxidation, and hydrolysis.

If there is no alkyne functionality on the protein, the alkyne functionality is added to the protein using techniques known to one of ordinary skills in the art. For example, a known ligand is modified to incorporate an alkyne. The ligand is bound to the target protein, preferably covalently. For example, if the protein has a thiol moiety, such as from a cysteine, the thiol is used to install an alkyne through alkynyl maleimide coupling; i.e, reacting the cysteine moiety with an alkynyl maleimide, wherein the alkynyl moiety contain 2-6 carbon atoms and 1 carbon-carbon triple bond and more preferably, the alkynyl moiety contains 2 or 3 carbon atoms and the carbon-carbon triple bond is in the 1-position of the alkynyl moiety.

### Examples

The following non-limiting examples further illustrate the present disclosure.

### Example 1

### Materials and Methods

Synthesis. General.

The coupling reaction was performed under an Ar atmosphere using dry solvents. All commercially available reagents were purchased from Sigma-Aldrich and were used as received. ¹H and ¹³C NMR spectra were recorded on Bruker instrument (400 or 500 MHz for ¹H and 100 or 125 MHz for ¹³C).

Acetal 1.

To a solution of 4-carboxybenzaldehyde (2.00 g, 13.3 mmol) in dry MeOH (40 ml) was added ammonium chloride (4.00 g, 74.8 mmol). The mixture was heated under reflux for 20 h. The solvent was evaporated under reduced pressure and the product was recrystallized from boiling hexane (2.0 g, 77%). ¹H NMR (500 MHz, MeOD) δ 8.15-8.01 (m, 2H), 7.62 (dd, J=21.9, 8.1 Hz, 2H), 5.50 (s, 1H), 3.43-3.32 (m, 6H).

Acetal 2.

Carbonyldiimidazole (88.7 mg, 0.54 mmol) and 1 (107.4 mg, 0.54 mmol) were dissolved in DCM. The mixture was stirred for 30 min at room temperature. To the solution was added azido-PEG₈-amine (200 mg, 0.45 mmol). After 5 h, the solvent was evaporated under reduced pressure. Product 2 was obtained by gravity column chromatography (basic alumina, 0%-5% MeOH/DCM) as an oil (210 mg, 75%)¹H NMR (500 MHz, CDCl₃) δ 7.87-7.75 (m, 2H), 7.51 (d, J=8.1 Hz, 2H), 5.43 (s, 1H), 3.73-3.56 (m, 34H), 3.42-3.35 (m, 2H), 3.32 (s, 6H); ¹³C NMR (126 MHz, CDCl3) δ 167.17, 167.15, 141.31, 134.66, 127.04, 127.02, 126.88, 102.35, 70.68-69.77, 52.61, 50.66, 39.80; MS (m/z): [M+H]⁺ calcd for C₂₈H₄₈N₄O₁₁ 617.33. found, 617.48

Trifluoroacetamide 3.

To a solution of 3-amino-1,2-propanediol (250 mg, 2.74 mmol) in THF, ethyl trifluoroacetate (2.33 g, 16.46 mmol) was added drop-wise. After 4 h, the solvent was evaporated. DCM was added to the oil and evaporated. This step was repeated two more times. Benzene was added and evaporated. This step was also repeated two more times. The resulting product was used without further purification to yield compound 3: ¹H NMR (500 MHz, CDCl₃) δ=3.3-3.6 (4H, m), 4.7-4.85 (1H, m); MS (m/z): [M]⁺ calcd for C₅H₈F₃NO₃ 186.05. found, 185.98.

Trifluoroacetamide 4.

Trifluoracetamide 4 was prepared from serinol (250 mg, 2.74 mmol) and ethyl trifluoroacetate (2.33 g, 16.46 mmol) as described for 3 to yield compound 4: ¹H NMR (500 MHz, CDCl₃) δ=3.45-3.55 (4H, m), 3.75-3.9 (1H, m), 4.75 (2H, t), 9 (1H, m); MS (m/z): [M]⁻ calcd for C₅H₈F₃NO₃ 186.05. found, 185.98

Trifluoroacetamide 5.

To a solution of 3 or 4 (191 mg, 1.022 mmol) in THF/Toluene (3/7), 2 (210 mg, 0.34 mmol) and p-toluene sulfonic acid.H₂O (13 mg, 0.068 mmol) were added. The mixture was heated to 100° C. The solvent was distilled to remove H₂O generated during the reaction and toluene added to maintain reaction volume as the reaction proceeded. After 4 h, the reaction was quenched with 50 μl of TEA. Product 5 was obtained by column chromatography (basic alumina, 0%-5% MeOH/DCM) as an oil. 5a (from 4, 180 mg, 72%): ¹H NMR (500 MHz, MeOD) δ 1H NMR (500 MHz, MeOD) δ 7.94-7.80 (m, 2H), 7.66-7.58 (m, 2H), 5.78-5.55 (m, HA 4.41-4.16 (m, 4H), 3.92-3.83 (m, 1H), 3.74-3.56 (m, 34H), 3.42-3.36 (m, 2H); MS (m/z): [M+H]⁺ calcd for C₃₁H₄₈F₃N₅O₁₂ 740.36. found 740.66. 5b (from 3, 160 mg, 64%): ¹H NMR (500 MHz, MeOD) δ 7.88 (dt, J=17.2, 7.8 Hz, 2H), 7.68-7.53 (m, 2H), 5.93 (d, J=86.5 Hz, 1H), 4.51-4.39 (m, 1H), 4.20 (ddd, J=53.7, 8.5, 6.8 Hz, 1H), 4.01-3.76 (m, 1H), 3.73-3.59 (m, 34H), 3.59-3.50 (m, 2H), 3.38 (dd, J=11.9, 6.6 Hz, 2H); MS (m/z): [M+NH₄]⁺ calcd for C₃₁H₄₈F₃N₅O₁₂ 757.36. found 757.55.

Amine 6.

To a solution of 5a or 5b (160 mg, 0.22 mmol) in MeOH/H₂O (7/3) K₂CO₃ (209.35 mg, 1.5149 mmol) was added. The reaction was heated at reflux for 2 h. After evaporating all the solvent, the product was purified by gravity column chromatography (basic alumina, 2%-10% MeOH/DCM) to yield an oil. 6a (from 5a, 134 mg, 82%): ¹H NMR (500 MHz, CDCl₃) δ 7.84 (dd, J=11.2, 5.0 Hz, 2H), 7.60-7.51 (m, 2H), 5.58-5.26 (m, 1H), 4.39-3.95 (m, 4H), 3.64 (dd, J=18.3, 3.7 Hz, 34H), 3.38 (d, J=4.4 Hz, 2H), 3.33-3.13 (m, 1H): [M+H]+ calcd for C₂₉H₄₉N₅O₁₁ 644.34. found 644.49. 6b (from 5b, 105 mg, 80%): ¹H NMR (400 MHz, CDCl₃) δ=7.82 (dt, J=18.4, 9.2 Hz, 2H), 7.53 (dd, J=16.4, 9.0 Hz, 2H), 6.05-5.78 (m, 1H), 4.40-4.23 (m, 1H), 4.14 (ddd, J=38.0, 16.3, 9.2 Hz, 1H), 3.92-3.72 (m, 1H), 3.70-3.55 (m, 34H), 3.37 (t, J=4.9 Hz, 2H), 3.05-2.81 (m, 2H). MS (m/z): [M+H]+ calcd for C₂₉H₄₉N₅O₁₁ 644.34. found 644.57.

Amide 7.

d-Biotin (50 mg, 0.205 mmol) and carbonydiimidazole (33 mg, 0.205 mmol) were dissolved in dried DMF. The mixture was stirred for 30 min. To the mixture, 6 was added and the reaction was stirred for 12 h at room temperature. The product was purified by gravity column chromatography (neutral alumina, 3%-7% MeOH/DCM) as an oil. Compound 7a (90 mg, 50%) 1H NMR (400 MHz, MeOD) δ 7.86 (t, J=6.7 Hz, 2H), 7.61 (dd, J=25.3, 8.3 Hz, 2H), 5.62 (dd, J=65.0, 13.7 Hz, 1H), 4.66-44.09 (m, 5H), 3.82 (s, 1H), 3.65 (m, 34H), 3.43-3.35 (m, 2H), 3.29-3.13 (m, 1H), 3.01-2.71 (m, 2H), 2.46-2.16 (m, 2H), 1.86-1.36 (m, 6H); MS (m/z): [M+H]+ calcd for C₃₉H₆₃N₇O₁₃S 870.42. found 870.38. Compound 7b (100 mg, 67%): ¹H NMR (400 MHz, MeOD) δ 7.88 (t, J=7.8 Hz, 2H), 7.60 (dd, J=21.2, 8.3 Hz, 2H), 6.00 (s, 1H), 5.83 (s, 1H), 4.53-4.30 (m, 2H), 4.23 (dt, J=12.1, 4.6 Hz, 1H), 4.10 (dd, J=8.2, 7.1 Hz, 1H), 3.89 (dt, J=13.8, 6.9 Hz, 1H), 3.83-3.72 (m, 1H), 3.65 (m, 34H), 3.55-3.41 (m, 2H), 3.41-3.37 (m, 2H), 3.23-3.10 (m, 1H), 2.95-2.84 (m, 1H), 2.70 (d, J=12.7 Hz, 1H), 2.35-2.17 (m, 2H), 1.85-1.35 (m, 6H); MS (m/z): [M+H]+ calcd for C₃₉H₆₃N₇O₁₃S 870.42. found 870.72. Compound 7c (70 mg, 61%): ¹H NMR (500 MHz, MeOD) δ 7.89 (t, J=7.6 Hz, 2H), 7.61 (dd, J=25.3, 8.2 Hz, 2H), 5.93 (d, J=81.6 Hz, 1H), 4.54-4.47 (m, 1H), 4.41-4.28 (m, 2H), 4.27-4.09 (m, 1H), 3.84 (ddd, J=14.9, 8.3, 6.0 Hz, 1H), 3.74-3.58 (m, 34H), 3.46 (dddd, J=22.8, 13.5, 11.9, 6.0 Hz, 4H), 3.26-3.13 (m, 3H), 2.99-2.91 (m, 1H), 2.73 (dd, J=12.7, 4.5 Hz, 1H), 2.24 (ddd, J=20.6, 14.2, 7.3 Hz, 4H), 1.72-1.31 (m, 12H); MS (m/z): [M+H]⁺ calcd for C₄₅H₇₄N₈O₁₄S: 982.5. found 983.5.

Biotin-PEG₁₀-N₃, 8.

Biotin-NHS (0.31 mmol, 107 mg) and O-(2-Aminoethyl)-0′-(2-azidoethyl)nonaethylene glycol (0.21 mmol, 110 mg) were dissolved in 1 mL dry DMF. DIEA (0.31 mmol, 56 μL) was added to the mixture, and the reaction was stirred for 16 h at room temperature. After evaporation of solvent, the product was precipitated with Et₂O. The chromatography (MeOH:EtOAc/1:1) yielded product 8. ¹H NMR (500 MHz, DMSO) δ 7.81 (t, J=5.5, 1H), 6.40 (br s, 1H), 6.34 (br s, 1H), 4.30 (m, 1H), 4.12 (m, 1H), 3.60 (m, 2H), 3.53 (m, 38H), 3.39 (t, J=5.1, 4H), 3.18 (q, J=5.8, 2H), 3.09 (dd, J=11.7, 7.3, 1H), 2.82 (dd, J=12.4, 5.1, 1H), 2.58 (d, J=12.4, 1H), 2.06 (t, J=7.4, 2H), 1.62 (dd, J=21.4, 7.9, 1H), 1.50 (dt, J=14.4, 7.5, 3H), 1.30 (m, 2H); MS (m/z): (MH+) calcd for C₃₂H₆₀N₆O₁₂S 753.4. found 753.4.

### Example 2

### A. Alkyne Functionalized BSA

To a solution of BSA (20 μM) in PBS, N-(1-propynyl)-maleimide (120 μM) was added. The mixture was gently agitated for 12 h in the dark. The excess maleimide was removed using an ultrafiltration spin filter (MWCO=3 kDa).

### B. Preparation of BSA Labeled with Biotin Probe

Alkyne functionalized BSA (50 μM) was mixed with biotin probe 7a or 7b (100 μM), BTTP (200 μM), CuSO₄ (100 μM), and sodium ascorbate (2.5 mM) for 1 h at room temperature. The regents were removed using an ultrafiltration spin filter (MWCO=3 kDa). The concentration of BSA was measured by Bradford assay (Pierce, Pierce Coomassie Plus protein assay, followed manufacturer's instruction).

### C. Cleavage Test

After coupling of BSA to the probe, the mixture was incubated with streptavidin-ultralink resin for 1 h at room temperature. The beads loaded with biotinylated BSA were spun at 1500 g for 3 min. The pelleted beads were washed with 2×0.1% SDS/PBS, 2×PBS, and 2×H₂O, sequentially. The beads were incubated with 1% TFA for 1 h at 37° C. The supernatant was collected by pelleting the beads. The beads were washed with 2×0.1% SDS/PBS, 2×PBS and the supernatants were combined with the washes. The combined solutions were concentrated using an ultrafiltration spin filter (MWCO=3 kDa) at 7000 g. Finally, the beads were boiled in sample loading buffer for 15 min.

### D. Analysis of Protein Capture and Release

Each protein sample was separated by 12% SDS-PAGE gel and transferred onto PDVF membrane (Bio Rad). The membrane was blocked with 4% BSA/TBST for 1 h at room temperature. (TBST: 50 mM Tris, 150 mM NaCl, 0.1% tween 20, pH 7.6). After washing the membrane with TBST three times, streptavidin conjugated with Alexa-488 (20 μg/ml) was added and it was gently agitated for 1 h at room temperature. The membrane was washed with TBST three times and was visualized using a Typhoon 9400 scanner (GE Healthcare).

To examine the efficiency of capture with the acetal biotin probes, BSA was used as a model protein. BSA has one cysteine on the surface and the thiol portion of the cysteine was employed to bond an alkyne moiety through N-alkynylmaleimide coupling in PBS. The alkyne-functionalized BSA was subjected to azide-alkyne cycloaddition with each of the biotin probes (FIG. 1A). The schematic of the synthesis is outlined herein below in Scheme 2. It is noted that compound 8 was prepared; compound 8 does not have the acetal moiety thereon.

In order to find effective cleavage conditions, each of the biotinylated BSA conjugates was incubated in 1% TFA at 37° C. with gentle agitation and aliquots were removed at each time point (30 minutes, 1 hour, and 2 hours). The biotin remaining on BSA was detected by streptavidin blot. The five-membered ring acetal 7b was successfully cleaved in 30 min. On the other hand, non-acetal biotin 8 and six-membered ring acetal 7a were stable to the cleavage conditions (FIG. 1A).

[chemical structure]

The five-membered acetal probe 7b was further tested to evaluate the efficiency of cleavage in the presence of streptavidin bead. The BSA conjugated to probe 7b or 8 was incubated with streptavidin beads for 1 hour at room temperature, and the beads were washed sequentially with 0.1% SDS/PBS, and water. The loaded beads were incubated with 1% TFA at 37° C. with gentle agitation. After 1 hour, the supernatant was collected and the first two washes were combined with it. After washing, the beads were boiled to elute BSA that was not released during the cleavage procedure. As shown in FIG. 1B, BSA-7b was successfully released from resin. However, a small amount of BSA conjugated to non-acetal probe 8 was released from the resin. In addition, a small amount of 8 was released under the cleavage conditions, although the probe linker remained intact as evidenced by the biotin signal in the streptavidin blot. Inefficient cleavage of 7b was due to limited solvent access to the acetal because the short linker between acetal and biotin places the acetal in close proximity to the biotin binding pocket on the streptavidin. The addition of various additives was investigated, e.g. SDS and guanidinium hydrochloride, to increase cleavage efficiency with limited success. Therefore, an extended linker with an additional seven atoms was introduced between the acetal and biotin by coupling NHS-LC-biotin with compound 6 (Scheme 1) to provide probe 7c.

The capture/cleavage procedures with streptavidin-ultralink resin were used to compare cleavage of 7c to 7b. However, the cleavage efficiencies of BSA-7b and BSA-7c were similar. Because the pore size of the bead can also affect solvent access to acetal and dissociation of the product aldehyde, the capture medium streptavidin-agarose beads were employed. The probe with the extended linker, 7c, was released more efficiently from the streptavidin-agarose bead complex than probe 7b which has a shorter linker. This result suggests that the combination of the extended linker and larger pore size are required to favor cleavage and dissociation of the acetal moiety.

Since the linker should be stable and efficiently capture in physiological condition, the cleavage test was performed in the presence of bacterial cell lysates.

Whole bacterial cell lysates mixed with BSA conjugated to probe 7b or 8 were incubated with streptavidin beads. After washing, the loaded beads were treated with 1% TFA at 37° C. for 1 hour. The cyclic acetal linker remained intact in the cell lysate and it was successfully used to capture BSA on the matrix and subsequently release it (FIG. 2).

The capture of another protein, RNase A that has a low molecular weight, 13.7 kDa was tested. RNase A has two free cysteines that were used to conjugate an alkyne handle via maleimide chemistry as described above for BSA. The alkyne was further conjugated with cleavable biotin probe 7c through azide-alkyne cycloaddition in the presence of Cu(I). RNase A-7c was captured from bacterial whole cell lysates and released as desired. However, the eluted protein was contaminated with streptavidin monomer that was released from the bead matrix during the cleavage step (FIG. 3A).

1M guanidinium hydrochloride was used to improve cleavage efficiency of cleaved protein release and suppressed the release of streptavidin. The use of guanidinium hydrochloride during cleavage was analyzed and compared its use to cleavage conditions for other cleavable biotin probes in order to determine whether the problem of streptavidin monomer release is widespread.

Streptavidin agarose beads were incubated separately under the following cleavage conditions: 5% Na₂S₂O₄ for 1 hour at 25° C., 2% of 2-mercaptoethanol for 1 hour at 25° C., 5% formic acid for 2 hours at 25° C., and 1M guanidine in 1% TFA at 37° C. for 1 hour. As shown in FIG. 3B, formic acid treatment also resulted in the release of streptavidin from agarose beads, whereas, reducing conditions did not. Inclusion of 1 M guanidine in the 1% TFA cleavage mixture suppressed non-specific release of the streptavidin monomer (FIG. 3A, lane 6 vs FIG. 3B, lane 4).

The effect of guanidine concentration on release and elution was tested. Two different concentrations, 1 M or 3 M guanidine hydrochloride, in combination with 1% TFA were tested. In both samples, suppression of streptavidin monomer release was observed. However, 3 M guanidine also releases some RNase A with the biotin probe still attached, indicating that the RNase A release is due to protein denaturation rather than acetal cleavage (FIG. 3C). The cleavage efficiency with BSA was tested since other proteins can be sensitive to 1M guanidine. 1M guanidine did not affect the release of BSA from the streptavidin resin.

In one embodiment, aldehyde tags can be used to modify cell surface proteins specifically since the aldehyde functionality is not typically present in proteins. Aldehydes readily react with a variety of aminooxy or hydrazide-functionalized molecules. Cleavage of the cyclic acetal linker 7 generates an aldehyde functionality on the tagged protein after purification. The cleaved BSA-7c was incubated with alkoxyamine-PEG-biotin for 4 hours, and the reaction mixture was directly analyzed by SDS-PAGE and streptavidin blot. After cleavage of the 7c acetal, no BSA biotin signal remained (FIG. 4, Lane 3). Upon reaction of the cleaved BSA with alkoxyamine-PEG-biotin, the biotin signal was restored (FIG. 4, Lane 4). Likewise, RNase A-7c underwent the analogous reaction sequence.

In the foregoing specification, the concepts have been described with reference to specific embodiments. However, one of ordinary skill in the art appreciates that various modifications and changes can be made without departing from the scope of the disclosure as set forth in the claims below. Accordingly, the specification is to be regarded in an illustrative rather than a restrictive sense, and all such modifications are intended to be included within the scope of disclosure.

Benefits, other advantages, and solutions to problems have been described above with regard to specific embodiments. However, the benefits, advantages, solutions to problems, and any feature(s) that may cause any benefit, advantage, or solution to occur or become more pronounced are not to be construed as a critical, required, or essential feature of any or all the present disclosure.

It is to be appreciated that certain features are, for clarity, described herein in the context of separate embodiments, may also be provided in combination in a single embodiment. Conversely, various features that are, for brevity, described in the context of a single embodiment, may also be provided separately or in any subcombination.

1. A compound of the Formula:
Q-L₁-X₂—X₃—R₂—R₃-L₂-Y I
wherein, [chemical structure]
X₁, X₂ and X₃ are independently absent, a bond, C(R₁)₂, NR₁, O, S, or CO;
each R₁ is the same or different and is H, or (C₁-C₁₀) alkyl;
L₁ is a bond, (C₂-C₅₀ alkyl), polyethylene glycol of the formula CH₂CH₂—(O—CH₂CH₂)n, wherein n is 1-30, a polypeptide of 1-15 amino acids of the formula AA₁-AAₙ₁, or a polypeptide of 1-15 amino acids of the formula: AAₘ-AA₁;
each AA is the same or different amino acid,
m and n₁ are independently 0-14.
R₂ is C₁-C₃₀ alkyl, disubstitued aryl, disubstituted arylalkyl, or
polyethylene glycol of the formula CH₂CH₂—(O—CH₂CH₂)ₙ₂ wherein n₂ is 1-30;
R₃ is [chemical structure]
L₂ is a bond, X₄-T-X₅, wherein T is an alkyl group of C₂-C₅₀, or a polyethylene glycol of the formula X₄—CH₂—CH₂—(O—CH₂—CH₂)n-X₅
X₄ is C(R₅)(R₆), CO;
X₅ is N(R₇), O, or S or a polypeptide of the formula AA₁-AAₙ₃
n₃ is 0-14
R₅, R₆, and R₇ and are independently H or C₁-C₁₀ alkyl, [chemical structure]
or an affinity tag peptide,
with the proviso that there can be no two adjacent oxygen, sulfur, nitrogen or carbonyl atoms, and when L₁ is a polypeptide of the formula AA₁-AAₙ₁, then X₂ is a bond and AA₁ is bonded to X₁ and AAₙ₁ is bonded to X₃ or when L₁ is a polypeptide of the formula: AAₘ-AA₁, then X₂ is absent and AA₁ is bonded to X₁ and AAₘ is bonded to X₃.

2. The compound of claim 1, wherein Q=N₃

[chemical structure]

3. The compound of claim 1, wherein,

[chemical structure]

4. The compound of the Formula:

[chemical structure]

5. A method of detecting the presence of a target molecule, comprising reacting a compound of claim 1 with a target molecule containing an alkyne moiety to form a compound-target complex; immobilizing the compound-target complex onto a stationary support; removing the compound-target complex from the support; and analyzing the target molecule.

6. The method of claim 5, wherein the target molecule is a protein.

7. A method of detecting the presence of a target molecule, comprising reacting a compound of claim 4 with a target molecule containing an alkyne moiety to form a compound-target complex; immobilizing the compound-target complex onto a stationary support; removing the compound-target complex from the support; and analyzing the target molecule.

8. The method of claim 7, wherein the target molecule is a protein.


SEQUENCE LISTING (3 sequences)

SEQ ID NO: 1 - protein, 8 aa, Artificial Sequence
  FLAG tag for affinity tagging of a peptide or small molecule
DYKDDDDK

SEQ ID NO: 2 - protein, 8 aa, Artificial Sequence
  Streptavidin tag for protein purification and identification
WSHPQFEK

SEQ ID NO: 3 - protein, 10 aa, Artificial Sequence
  c-Myc Tag for protein purification and identification
EQKLISEEDL